(19) United States
(12) Patent Application Publication
(10) Pub. No.: US 2023/0181881 A1
(43) Pub. Date: Jun. 15, 2023

(54) STEERABLE MEDICAL DEVICES

(71) Applicants: Boston Scientific Scimed, Inc., Maple Grove, MN (US); Boston Scientific Medical Device Limited, Galway (IE)

(72) Inventors: Deepak Kumar SHARMA, Muzaffarnagar (IN); Charles GIBSON, Malden, MA (US); James SCUTTI, Norwell, MA (US); Sharath Kumar G, Kanakapura (IN); Hitendra PUROHIT, Vadodara (IN); Juan Pablo ORTIZ GARCIA, Heredia (CR); Shrikant Vasant RAUT, Mumbai (IN)

(73) Assignees: Boston Scientific Scimed, Inc., Maple Grove, MN (US); Boston Scientific Medical Device Limited, Galway (IE)

(21) Appl. No.: 18/079,919

(22) Filed: Dec. 13, 2022

Related U.S. Application Data
(60) Provisional application No. 63/265,389, filed on Dec. 14, 2021.

(51) Int. Cl. A61M 25/09

(52) U.S. Cl. CPC A61M 25/09041; A61M 2025/09058; A61M 2025/09116; A61M 2025/09125

(57) ABSTRACT

A medical device may comprise a shaft, an articulation wire extending through at least a portion of the shaft, and a handle, including a handle body having an outer surface that defines a channel. A distal portion of the shaft may be received within a first portion of the channel. A distal portion of the articulation wire may be received within a second portion of the channel. A control member may be movable relative to the handle body to move the distal portion of the articulation wire relative to the distal portion of the shaft.

### CROSS-REFERENCE TO RELATED APPLIATION(S)

This application claims the benefit of priority of U.S. Provisional Application No. 63/265,389, filed Dec. 14, 2021, the entirety of which is incorporated by reference herein.

### TECHNICAL FIELD

The disclosure relates generally to devices, systems, and methods for steerable medical devices. More specifically, aspects of the disclosure pertain to devices, systems, and/or methods for steerable guidewires.

### BACKGROUND

An operator may insert tools through a working channel of a medical device during procedures such as endoscopy, colonoscopy, and/or Endoscopic Retrograde Cholangiopancreatography (“ERCP”). In such procedures, an operator may desire to position an instrument or device (such as a tool) at a particular position, at or distal to a distal tip of the medical device. In order to position an instrument or device in a desired position, an operator may utilize a guidewire. An operator may pass a guidewire through a working channel of an endoscope (or through a lumen of another device, such as a guide catheter or tome), and then use the guidewire in order to position the instrument or device. For example, an operator may slide the instrument, such as a snare, or device, such as a stent, over the guidewire. Guidewires may also facilitate exchange of instruments and devices in an efficient fashion. An operator may remove and replace an instrument or device, guiding the new instrument or device to the same location by using the guidewire.

Guidewires may also be utilized in vascular applications. For example, an operator may utilize a guidewire in order to position a stent, catheter, or other structure. Guidewires may similarly be used in urological procedures (e.g., to place a ureteral stent or a catheter). Positioning a guidewire in a desired position is important for each of these procedures, as well as others involving guidewires. Therefore, a need exists for devices, systems, and methods for steerable medical devices, including steerable guidewires.

### SUMMARY

In one example, a medical device comprises a shaft, an articulation wire extending through at least a portion of the shaft, and a handle, including a handle body having an outer surface that defines a channel. A distal portion of the shaft may be received within a first portion of the channel. A distal portion of the articulation wire may be received within a second portion of the channel. A control member may be movable relative to the handle body to move the distal portion of the articulation wire relative to the distal portion of the shaft.

Any of the medical devices and methods disclosed herein may have any of the following features. The articulation wire may be a first articulation wire, and the control member may be a first control member, and the device may further comprise a second articulation wire extending through the at least the portion of the shaft. A proximal portion of the second articulation wire may be removably received within a third portion of the channel. A second control member may be movable relative to the handle body to move the distal portion of the second articulation wire relative to the distal portion of the shaft. Each of the second portion of the channel and the third portion of the channel may be proximal to the first portion of the channel. The channel may have an approximately Y-shape. A wire fixation body may be connected to the control member. The wire fixation body may define a first slot in communication with the second portion of the channel. The articulation wire may be removably received within the first slot. The control member may be a first control member. The device may further include a second control member. Movement of the second control member relative to the handle body may change a width of the first slot. A shaft fixation body may define a second slot in communication with the first portion of the channel. Movement of the second control member relative to the handle body may change a width of the second slot. Movement of the second control member in a first direction relative to the handle body may widen the first slot and the second slot so that the shaft is removable from the second slot and the articulation wire is removable from the first slot. Movement of the second control member in a second direction relative to the handle body may narrow the first slot and the second slot so that the articulation wire is fixedly secured relative to the first slot and the shaft is fixedly secured relative to the second slot. A first lumen may extend proximally from the wire fixation body, and a second lumen may extend proximally from the shaft fixation body. The shaft fixation body may be distal to the wire fixation body. The second control member may include a slider. The first control member may be disposed on a first side of the handle. The second control member may be disposed on a second side of the handle. The second side may be different from the first side. The channel may be on the first side of the handle. The articulation wire may extend proximally of a proximal end of the shaft.

In another example, a medical device may comprise a shaft, a first articulation wire extending through at least a portion of the shaft, a second articulation wire extending through at least the portion of the shaft, and a handle, including a first wire fixation body defining a first slot. A proximal end of the first articulation wire may be received within the first slot. A second wire fixation body may define a second slot, wherein a proximal end of the second articulation wire is received within the second slot. The device may further comprise a control member. Movement of the control member may change a first width of the first slot and a second width of the second slot.

Any of the medical devices and methods disclosed herein may include any of the following features. A shaft fixation body may define a third slot. The shaft may be received within the third slot. Movement of the control member may change a third width of the third slot. The control member may be a first control member. The medical device may further comprise a second control member configured to move the first wire fixation body, thereby moving the proximal end of first articulation wire relative to the shaft, and a third control member configured to move the second wire fixation body, thereby moving the proximal end of the second articulation wire relative to the shaft.

An exemplary method of using a medical device may comprise inserting a proximal end of a shaft into a first portion of a channel defined by a body of a handle of the medical device, inserting a proximal end of a first articulation wire into a second portion of the channel, inserting a proximal end of a second articulation wire into a third portion of the channel, and moving a control member in a first direction relative to the body, thereby securing the shaft, the first articulation wire, and the second articulation wire to the handle.

Any of the methods disclosed herein may include any of the following steps. The method may include moving a control wire in a second direction relative to the body, thereby releasing the shaft, the first articulation wire, and the second articulation wire from the handle, and removing the shaft, the first articulation wire, and the second articulation wire from the handle.

A further exemplary medical device may include a handle and a shaft. In a first configuration, the shaft may be connected to the handle, such that manipulation of a control member of the handle articulates a distal portion of the shaft. In a second configuration, the shaft may be disconnected from the handle. The medical device may be configured such that the device may be transitioned from the first configuration to the second configuration, and from the second configuration to the first configuration. The device may thereby allow the shaft to be repositioned from a first position within a body to a second position within the body during a medical procedure.

Any of the medical devices and methods disclosed herein may include any of the following features. The shaft may include a guidewire. The guidewire may be configured to guide medical devices within a body lumen. The handle may facilitate repositioning the guidewire from the first position to the second position. The control member may facilitate repositioning the guidewire from the first position to the second position.

Another exemplary method of using a medical device may include inserting a shaft into a body of a subject, using a handle to navigate a distal portion of the shaft to a first location within the body, removing the handle from the shaft, reattaching the handle to the shaft after removing the handle, and using the handle to navigate the distal portion of the shaft to a second location within the body.

Any of the methods disclosed herein may include any of the following steps. The method may further include, after removing the handle from the shaft and before reattaching the handle to the shaft, passing one or more medical devices along the shaft. The shaft may include a guidewire. The method may include removing the handle from the shaft after reattaching the handle to the shaft.

It may be understood that both the foregoing general description and the following detailed description are exemplary and explanatory only and are not restrictive of the invention, as claimed. As used herein, the terms “comprises,” “comprising,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” The term “distal” refers to a direction away from an operator/toward a treatment site, and the term “proximal” refers to a direction toward an operator. The term “approximately,” or like terms (e.g., “substantially”), includes values +/- 10% of a stated value.

### BRIEF DESCRIPTION OF THE DRAWINGS

The accompanying drawings, which are incorporated in and constitute a part of this specification, illustrate examples of this disclosure and together with the description, serve to explain the principles of the disclosure.

FIG. 1 depicts an exemplary steerable medical device.

FIGS. 2A-3J depict aspects of an exemplary handle for the medical device depicted in FIG. 1.

FIGS. 4A-5D depict aspects of exemplary shafts for a medical device.

FIG. 6 depicts an alternative steerable medical device.

FIGS. 7A-7B depict a further alternative steerable medical device.

FIGS. 8A-8B depict a further alternative steerable medical device.

FIG. 9 depicts aspects of an exemplary shaft for use with one of the exemplary medical devices depicted in FIG. 1-8B.

FIGS. 10A-10C depict another steerable medical device.

FIGS. 11A-11B depict exemplary shafts for use with any of the medical devices depicted in FIGS. 1-3J, 6-8B, and 10A-10C.

### DETAILED DESCRIPTION

A medical device may include a handle and a steerable shaft. The shaft may include, for example, a steerable guidewire. Although the disclosure may reference guidewires used in conjunction with endoscopic, colonoscopic, and/or ERCP procedures, it will be appreciated that the guidewires may also be utilized for other types of procedures, including vascular or urologic procedures. The medical devices disclosed herein may have three degrees of freedom for movement. The shaft may be articulated (i.e. bent or deflected) in one or more directions. Furthermore, the shaft may be rotatable and movable proximally and distally. The handle may be removable from the shaft so as to facilitate use of the guidewire to guide tools or medical devices after the guidewire is positioned. The shaft may include two lumens with one or two articulation wires, either or both of which may be utilized to achieve a desired steering/articulation of the shaft. The medical devices disclosed herein may overcome limitations to accessing complex anatomies and resolve or assist with navigation challenges. The disclosed medical devices may eliminate the high learning curve associated with other devices. The disclosed medical devices may supplant the multiple guidewires that are currently used, depending on anatomy and the type of medical procedure, thereby streamlining the types of guidewires that a medical provider must stock and limiting potential for confusion surrounding different types of guidewires. The disclosed devices may be compatible with robotics platforms. Furthermore, procedures may require accessing a first location in an anatomy, followed by one or more further locations, location within the same area of the anatomy. The disclosed, steerable devices may allow access to multiple locations within the area of the anatomy, without inserting and removing a first guidewire and re-navigating to additional locations. Thus, the disclosed devices may avoid needing multiple guidewires to be placed in multiple locations in the anatomy. Any of the handles disclosed herein may be removed and/or replaced with the same handle or with an alternate handle. Similarly, the shafts disclosed herein may be utilized with any of the disclosed handles. Thus, during a procedure, an operator may use different handles at different portions of the procedure or may use a shaft without a handle.

Although the disclosure may refer at different points to one of a duodenoscope or an endoscope, it will be appreciated that, unless otherwise specified, duoendoscopes, endoscopes, colonoscopes, ureteroscopes, bronchoscopes, laparoscopes, sheaths, catheters, or any other suitable delivery device or medical device may be used in connection with the guidewires and methods of the disclosure.

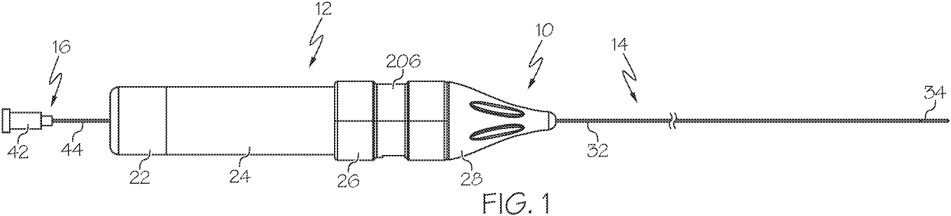

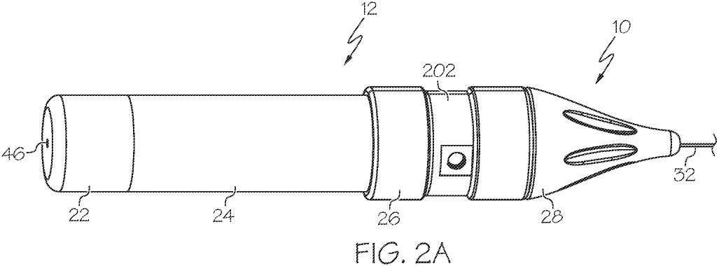

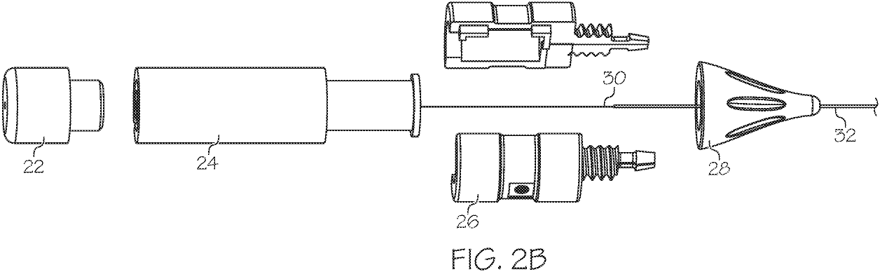

FIG. 1 depicts an exemplary medical device 10. FIGS. 2A-5D depict aspects of medical device 10 that can be used together in combination or in the alternative to one another. As shown in FIG. 1, medical device 10 may include a handle 12 and a shaft 14. Shaft 14 may have any suitable outer width (e.g., diameter). For example, an outer width (e.g., diameter) of shaft 14 may be similar to or the same as a typical guidewire. Exemplary outer diameters may range from approximately .010 inches to approximately .04 inches or more particularly from approximately .014 inches to approximately .032 inches. An outer diameter may be constant along a length of shaft 14 or may vary.

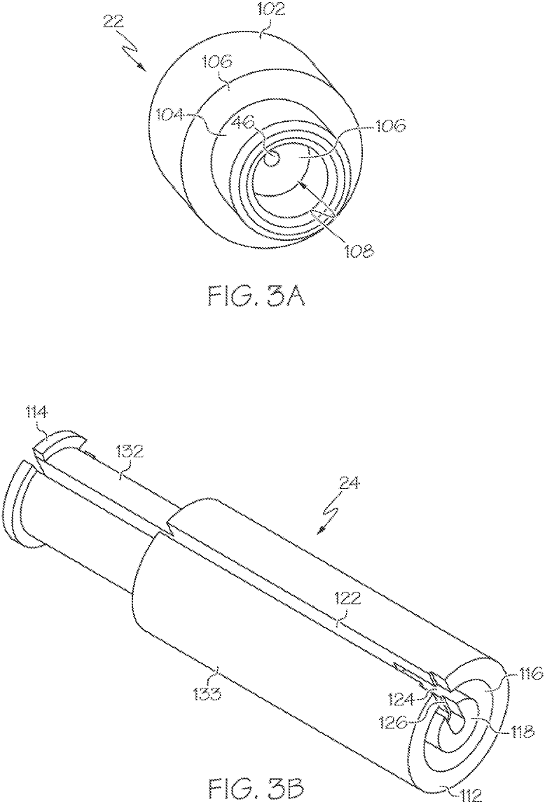

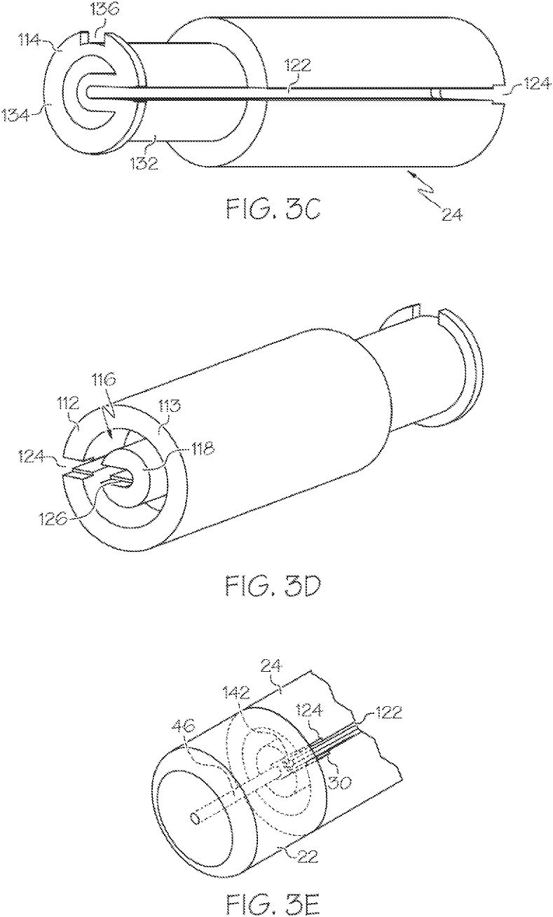

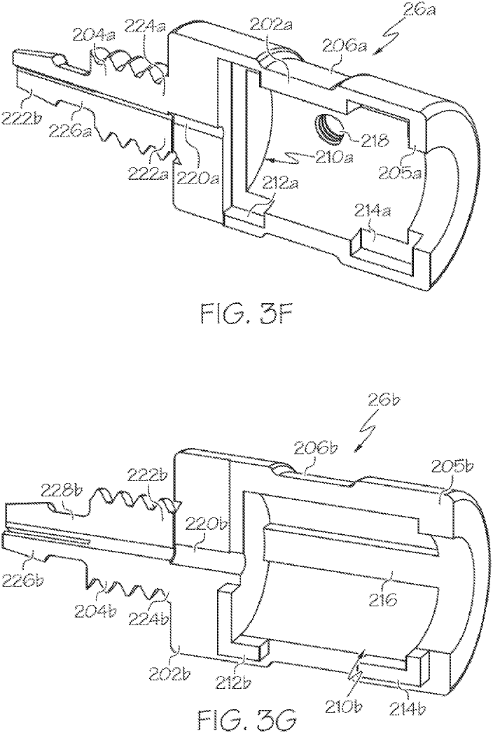

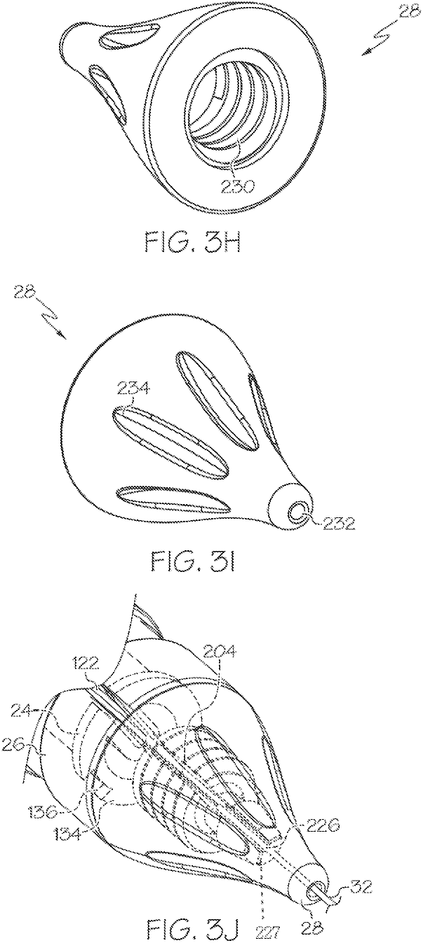

Handle 12 may include various bodies, including a cap 22, a plunger 24, a slider 26, and a collet 28. Shaft 14 may include a proximal section 32 and a distal section 34 (distal section 34 being distal to proximal section 32). A contrast delivery device 16 may be used along with medical device 10. Contrast delivery device 16 may include a reservoir 42 and a cannula 44. Contrast delivery device 16 may be actuated (e.g., via a plunger), in order to cause contrast to pass from reservoir 42 through cannula 44. Contrast delivery device 16 may include a gun-like handle, which may attach to a syringe, to provide for greater precision and control while delivering contrast. As explained in further detail below, cap 22 may have a contrast lumen 46 (see FIGS. 2A, 3A, and 3E) formed therein for receiving the contrast from the contrast delivery device 16.

FIGS. 2A-3J depict further details of handle 12. FIG. 2A shows a perspective view of handle 12, and FIG. 2B shows handle 12 in a state of partial disassembly in order to depict its components and operation more clearly. FIGS. 3A-3J depict components of handle 12. As shown with particular reference to FIGS. 2B and 3A, cap 22 may include a cylindrical body 102. An annular extension 104 may extend distally from a distal surface 106 of cylindrical body 102. Annular extension 104 may have a smaller radius than cylindrical body 102 and may be coaxial with cylindrical body 102. Annular extension 104 may have a circular cross section (in a direction perpendicular to a central longitudinal axis of cap 22 and medical device 10). Walls of annular extension 104 may have a constant or a variable thickness. Distal surface 106 and radially inner walls of annular extension 104 may define a cavity 108. Annular extension 104 may facilitate assembling cap 22 with plunger 24, as described below.

Contrast lumen 46 may extend coaxial with or parallel to the central longitudinal axis of cap 22. Contrast lumen 46 may have a proximal opening on a proximal surface of cap 22 and may have a distal opening on distal surface 106. Annular extension 104 may surround the distal opening of contrast lumen 46, and contrast lumen 46 may be coaxial with or parallel to a central longitudinal axis of annular extension 104.

As shown particularly in FIGS. 2A-2B and 3B-3D, plunger 24 may have an approximately cylindrical shape extending between a proximal end 112 and a distal end 114, such that a cross-section of plunger 24 (perpendicular to a longitudinal axis of plunger 24) is circular or approximately circular. Distal end 114 (shown particularly in FIGS. 3C and 3D) may include an annular recess 116. Annular recess 116 and annular extension 104 may be dimensioned so that annular extension 104 fits within annular recess 116 when medical device 10 is assembled, as shown in FIG. 3E. Steps for assembling medical device 10 will be described in further detail below. A center portion 118 of proximal end 112 may be surrounded by annular recess 116. Radially outward of annular recess 116, proximal end 112 may include an annular perimeter 113. Center portion 118 and cavity 108 may be dimensioned so that center portion 118 fits within cavity 108 when medical device 10 is assembled. Surfaces of cap 22 and plunger 24 may have an interference fit so that, when medical device 10 is assembled, cap 22 remains on plunger 24. An operator may be able to manually remove cap 22 from plunger 24.

A slit or slit (i.e., a slot) 122 may extend through a radially outward wall of plunger 24. Slit 122 may extend along an entire length of plunger 24, from proximal end 112 to distal end 114. Slit 122 may have a closed, radially-inward inner end, such that one or more walls of plunger 24 define surfaces of slit 122. A depth of slit 122 may be approximately half as wide as a width of plunger 24 (where the width is perpendicular to the longitudinal axis of plunger 24). If plunger 24 has a circular cross-section, the depth of slit 122 may be approximately equal to a radius of the cross-section. As shown in FIG. 3D in particular, slit 122 may extend through annular perimeter 113, across a width of annular recess 116, into center portion 118. At distal end 114, slit 122 may terminate (have a closed end) within center portion 118. The closed end of slit 122 may be rounded. For example, the closed end of slit 122 may have an approximately semicircular shape. Portions of slit 122 may have different dimensions. For example, at a proximal end 124 of slit 122 (at proximal end 112 of plunger 24), slit 122 may be wider. Slit 122 may transition from a greater to a smaller width. For example, surfaces of slit 122 may define a ledge 126. A portion of ledge 126 may be defined by surfaces of annular perimeter 113, and a portion of ledge 126 may be defined by surfaces of center portion 118. As discussed in further detail before, ledge 126 may facilitate placement of an articulation wire 30 (shown in FIG. 2B).

A distal portion of plunger 24 may include a neck portion 132 that has a narrower width than a proximal portion 133 of plunger 24. Along a longitudinal axis of plunger 24, neck portion 132 may be shorter than proximal portion 133. Distal end 114 may include a flange 134. Flange 134 may have a greater width than neck portion 132. Flange 134 may have a smaller width than proximal portion 133 or may have an approximately equal width to proximal portion 133 or a greater width than proximal portion 133. A notch 136 may be formed in flange 134. Notch 136 may extend through an entire length (along a longitudinal axis of plunger 24) of flange 134. As discussed below, notch 136 may mate with a ridge 216 of slider 26.

As shown in FIGS. 2B and 3F-3G, slider 26 may include two portions (e.g., halves or approximate halves)-first slider portion 26a and second slider portion 26b. Slider 26 may include a slider body 202. First slider portion 26a may include a first slider body 202a, and second slider portion 26b may include a second slider body 202b, which together form slider body 202. Slider 26 may also include a locknut screw 204 (see FIG. 3J). First slider portion 26a may include a first locknut screw portion 204a, and second slider portion 26b may include a second locknut screw portion 204b, which together may form locknut screw 204.

First slider body 202a of first slider portion 26a may define a first cavity 210a. Second slider body 202b of second slider portion 26b may define a second cavity 210b. When first slider portion 26a and second slider portion 26b are combined, first cavity 210a and second cavity 210b may combine to slidably receive a portion of plunger 24. For example, first cavity 210a and second cavity 210b may combine to receive at least a portion of neck portion 132 of plunger 24. Outer surfaces of first slider body 202a and second slider body 202a may each include a recessed portion 206a, 206b, respectively, which combine to form a recessed portion 206. Recessed portion 206 may facilitate gripping by an operator.

At a proximal end of first slider portion 26a, first slider body 202a may include a first rim 205a. Similarly, at a proximal end of second slider portion 26b, second slider body 202b may include a second rim 205b. Each of first rim 205a and second rim 205b may have an approximately semicircular shape so that, when first slider portion 26a and second slider portion 26b are combined, first rim 205a and second rim 205b form an approximately circular rim, defining an approximately circular proximal opening. The proximal opening may be dimensioned so as to at least partially surround neck portion 132. First rim 205a and second rim 205b may serve to retain flange 134 of plunger 24, to inhibit relative movement between slider 26 and plunger 24 past first rim 205a and second rim 205b. Flange 134 may have a greater width (e.g., diameter) than an opening defined by first rim 205a and second rim 205b. Therefore, slider 26 may not be able to move distally past flange 134 because an annular ledge defined by first rim 205a and second rim 205b may encounter flange 134.

When combined, distal ends of first slider body 202a and second slider body 202b, may define a small opening to a lumen (discussed in further detail below) for receiving an articulation wire/and or a portion of shaft 14. The opening may have a smaller diameter than flange 134, such that distal ends of first slider body 202a and second slider body 202b inhibit relative movement between plunger 24 and slider 26 past a distal end of the cavity defined slider body 202.

As shown in FIGS. 3F and 3G, first slider portion 26a and second slider portion 26b may include various features to facilitate mating with one another and other portions of medical device 10. For example, second slider portion 26b may include a first protrusion 212b and a second protrusion 214b, which may be received by corresponding first groove 212a and second groove 214a of first slider portion 26a. As shown in FIG. 3G, first protrusion 212b and second protrusion 214b may each have an “L” shape. The dimensions of the legs of the “L” shape of first protrusion 212b and second protrusion 214b may vary from one another and may have any suitable shape. One of the legs of each of first protrusion 212b and second protrusion 214b may extend approximately along a longitudinal axis of medical device 10 (and slider 26). A second of the legs of each of first protrusion 212b and second protrusion 214b may extend in an approximately radial direction (perpendicular to the longitudinal axis of medical device 10). First groove 212a and second groove 214a may have shapes so as to correspond to first protrusion 212b and second protrusion 214b. First protrusion 212b and second protrusion 214b may have a friction fit with first groove 212a and second groove 214a, such that first protrusion 212b is retained within first groove 212a and second protrusion 214b is retained within second groove 214a, absent application of a force sufficient to separate them. A human hand may exert a sufficient force to separate first protrusion 212b from first groove 212a and second protrusion 214b from second groove 214a by pulling apart second slider portion 26b and first slider portion 26a. Numbers and arrangements of protrusions 212b, 214b and grooves 212a, 214a are merely exemplary, and alternative arrangements may be used.

Ridge 216 may have a complementary shape to and mate with notch 136, such that ridge 216 rides along notch 136, as slider 26 moves proximally or distally relative to plunger 24. An interaction between ridge 216 and notch 136 may inhibit slider 26 from rotating about a longitudinal axis of medical device 10, relative to plunger 24. As shown in FIG. 3G, ridge 216 may extend longitudinally along a length of second cavity 210b, protruding from an inner surface of second slider body 202b. A radially inner surface of ridge 216 may have a radius of curvature that is approximately the same as a radius of curvature of a radially inner surface of notch 136.

First slider body 202a (or second slider body 202b) may include a threaded hole 218 for receiving a screw, such as a grub screw. The grub screw (not shown) may be adjusted in order to adjust a stroke of slider 26 along plunger 24. It may be desirable to adjust (e.g., shorten) a stroke of slider 26 so as to avoid over-tensioning articulation wire 30. For example, when a grub screw is inserted through hole 218, the grub screw may interact with flange 134 to limit slider 26 from sliding over plunger 24 beyond the grub screw.

When first slider portion 26a and second slider portion 26b are combined, first locknut screw portion 204a and second locknut screw portion 204b may have threads that align with one another. First locknut screw portion 204a may have a first free proximal end portion 222a. First free proximal end portion 222a may be separated from a distal outer surface of first slider body 202a by a gap. First locknut screw portion 204a may also have a first fixed proximal end portion 224a. First fixed proximal end portion 224a may be fixed relative to the distal outer surface of first slider body 202a. First free proximal end portion 222a may form approximately half of first locknut screw portion 204a. Alternatively, first free proximal end portion 222a may constitute a different percentage of first locknut screw portion 204a. Second locknut screw portion 204b may similarly have a second free proximal end portion 222b and a second fixed proximal end portion 224b. Second free proximal end portion 222b may be separated from a distal outer surface of second slider body 202b by a gap. Second fixed proximal end portion 224b may be fixed relative to the distal outer surface of second slider body 202b. Second free proximal end portion 222b may form approximately half of second locknut screw portion 204b. Alternatively, second free proximal end portion 222b may constitute a different percentage of second locknut screw portion 204b. When first slider portion 26a and second slider portion 26b are combined, first free proximal end portion 222a may align with second free proximal end portion 222b, and first fixed proximal end portion 224a may align with second fixed proximal end portion 224b.

First slider portion 26a may include a first collar portion 226a, and second slider portion 26b may include a second collar portion 226b. A first bridge portion 228a may extend between first locknut screw portion 204a and first collar portion 226a. Similarly, a second bridge portion 228b may extend between second locknut screw portion 204b and second collar portion 226b. Bridge portions 228a, 228b may have a narrower width than the respective locknut screw portions 204a, 204b and the respective collar portions 226a, 226b. Each of first collar portion 226a and second collar portion 226b may have an outer surface that tapers in a distal direction. One or both of first collar portion 226a and second collar portion 226b may define slits 227 (see FIG. 3J). For example, first collar portion 226a and second collar portion 226b may together define three slits 227 or an alternative number of slits 227 As explained in further detail below, slits 227 may divide first collar portion 226a and second collar 226b portions into pieces that may flex radially inward or outward to grip or release, respectively shaft 14. When first slider portion 26a and second slider portion 26b are combined, first collar portion 226a and second collar portion 226b may combine to form a collar 226.

A first channel 220a may extend longitudinally through a distal wall of first slider body 202a, first locknut screw portion 204a, and first collar portion 226a. Similarly, second channel 220b may extend longitudinally through a distal wall of second slider body 202b, second locknut screw portion 204b, and second collar portion 226b. When first slider portion 26a and second slider portion 26b are combined, the channels 220a, 220b may align with one another to define a lumen. The lumen may be in communication with the cavity defined by slider body 202 (i.e., the combination of first cavity 210a and second cavity 210b). The lumen may be wider (e.g., have a greater diameter) at a proximal end of the lumen, and narrower (e.g., have a smaller diameter) at a distal end of the lumen.

FIGS. 3H-3I depict details of collet 28. FIG. 3J shows collet 28 positioned on a distal end of slider 26. Collet 28 may have a shape that tapers distally. A proximal end of collet 28 may have a cross-sectional width (e.g., a diameter) that is approximately equal to a cross-sectional width (e.g., a diameter) of a distal end of slider body 202. A distal end of collet 28 may have a cross-sectional width (e.g., a diameter) that is between a cross-sectional width (e.g., diameter) of a distal end of collar 226 and cross-sectional width (e.g., diameter) of proximal section 32 of shaft 14.

A proximal inner surface 230 of collet 28 may define threads configured to interact with threads of locknut screw 204. A distal inner surface of collet 28 may define a lumen 232, having an opening at a distal end of collet 28, for receiving shaft 14. An outer surface of collet 28 may include detents 234. Detents 234 may have an oval or elliptical shape and may extend along in a longitudinal direction.

With particular reference to FIGS. 2B, 3E, and 3J, assembly of handle 12 will now be discussed. Operation of handle 12 will be discussed in further detail following further discussion of features of shaft 14. The following steps outlined below may be performed in various orders, unless otherwise noted below. As shown in FIG. 2B, articulation wire 30 may extend proximally from a proximal end of shaft 14. A proximal end of articulation wire 30 may have a ball 142 (or another type of enlarged portion having a greater cross-sectional size than a cross-sectional size of an adjacent portion of articulation wire 30, such as an increased diameter portion). During assembly by a manufacturer or a user, ball 142 may be inserted into proximal end 124 of slit 122. Ball 142 may have a width (e.g. a diameter) that is greater than a width of slit 122 distal to ledge 126. But proximal end 124 of slit 122 may be wider than ball 142. Therefore, ball 142 may rest on ledge 126 of slit 122, as shown in FIG. 3E. Articulation wire 30 may be inserted into slit 122, such that it extends longitudinally along slit 122.

After articulation wire 30 is positioned within slit 122, first slider portion 26a and second slider portion 26b may be fit together such that first groove 212a aligns with first protrusion 212b and second groove 214a aligns with second protrusion 214b. In alternatives, first slider portion 26a and second slider portion 26b may be connected via a hinge, which may be closed during assembly. Shaft 14 may be positioned so that at least a portion of proximal section 32 extends through at least a portion of the lumen defined by first channel 220a and second channel 220b. Proximal section 32 may extend at least partially through locknut screw 204 and may extend at least partially through a distal end of slider body 202.

Shaft 14 may be inserted through lumen 232 of collet 28. For example, prior to positioning articulation wire 30 within slit 122 of plunger 24, a proximal end of articulation wire 30 may be passed proximally through the distal opening of lumen 232 of collet 28. Alternatively, before or after positioning articulation wire 30 within slit 122 of plunger 24, a distal end of shaft 14 may be passed distally through a proximal opening of lumen 232. After first slider portion 26a and second slider portion 26b are positioned about plunger 24, collet 28 may be screwed onto locknut screw 204, such that threads of lumen 232 engage with threads of locknut screw 204. Collet 28 may exert a radially inward force on locknut screw 204, thereby gripping proximal section 32. In particular, first free proximal end portion 222a and second free proximal end portion 222b may press radially inward on proximal section 32. Additionally or alternatively, collet 28 may exert a radially inward pressure on portions of collar portions 226a, 226b between slits 227 so that collar portions 226a, 226b additionally grip onto proximal section 32. Thus, portions of handle 12 may grip proximal section 32 of shaft 14, removably marrying shaft 14 and handle 12 and allowing for steering of shaft 14, as described below.

In alternatives to collet 28, a cam-locking latch may be hinged to first slider body 202a or second slider body 202b. The cam-locking latch may be curved to match a shape of a distal end of handle 12 and may include a cam profile on an interior surface (facing an interior of handle 12). Upon swinging the latch into place over the distal end of handle 12 (including first slider body 202a and second slider body 202b), the latch may pinch collar portions 226a, 226b so that it grips proximal section 32 of shaft 14 and also tightly retains first slider body 202a and second slider body 202b. Other alternative structures may also be used that sufficiently grip proximal section 32 of shaft 14, such that when articulation wire 30 is moved, shaft 14 does not move along with it and, instead, articulation wire 30 moves relative to shaft 14.

After articulation wire 30 is positioned within slit 122, cap 22 may be positioned on a proximal end of plunger 24. Center portion 118 of cap 22 may be inserted into cavity 108 of plunger 24 when medical device 10 is assembled. Cap 22 may secure ball 142 on ledge 126 of plunger 24.

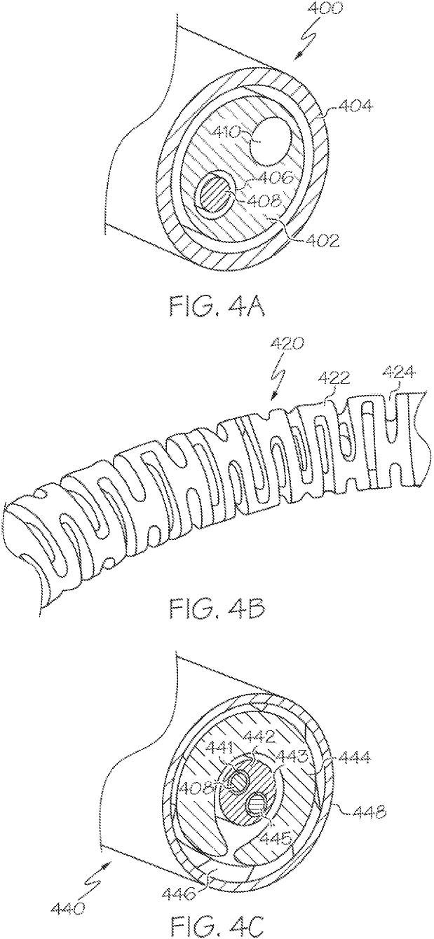

FIGS. 4A-4C depict exemplary features of shafts that may be used as a proximal section 32 of shaft 14. Materials for proximal section 32 and distal section 34 of shaft 14 may be chosen to optimize navigation of a body lumen and to define points on shaft 14 at which articulation may begin.

Proximal section 32 of shaft may have any suitable length along a longitudinal axis of shaft 14. For example, a length of proximal section 32 may be approximately 0.6 m. As shown in FIG. 4A, a shaft portion 400 may include a core inner shaft 402 and an outer shaft 404. Inner shaft 402 may be molded, extruded, or otherwise formed so that it is a single, integral structure that defines one or more openings that form lumens. For example, as shown in FIG. 4A, inner shaft 402 may define two lumens. A first lumen 406 may house an articulation wire 408 (which may be used as articulation wire 30 discussed above). A second lumen 410 may be used to house a second articulation wire (not shown), or a cable for a sensor (such as an electromagnetic sensor, described below). Additionally or alternatively, second lumen 410 may, directly or via a separate tube within second lumen 410, transmit contrast (e.g., from contrast delivery device 16). At least first lumen 406 may extend approximately parallel to a central longitudinal axis of shaft portion 400. At least first lumen 406 may be offset (i.e., oblique) from a center longitudinal axis of shaft portion 400. Second lumen 410 may also be parallel to and offset from the central longitudinal axis of shaft portion 400. Inner shaft 402 may include, for example, polytetrafluoroethylene (“PTFE”) and/or other suitable materials. Inner shaft 402 may have any suitable outer diameter. For example, for a shaft 14 having an outer diameter of approximately 0.03 inches, inner shaft 402 may have a diameter of approximately 0.024 inches.

Outer shaft 404 may be molded, extruded, or constructed by alternative methods. Outer shaft 404 may be formed atop of inner shaft 402 or separately from inner shaft 402. Outer shaft 404 may be constructed so as to have sufficient stiffness to allow for longitudinal movement of shaft 14 (e.g., by pushing and pulling handle 12 proximally and distally) and rotation of shaft 14 (e.g., by rotating handle 12 about a longitudinal axis). As shown in FIG. 4B, outer shaft 404 may include at least a Nitinol portion 420 and may have patterns of cut-outs 424 as to provide flexibility and facilitate bending of shaft 14. As shown in FIG. 4B, cut-outs 424 may alternate with segments 422 of material in order to provide flexibility. Cut-outs 424 and segments 422 may be arranged in any suitable way to provide a desired flexibility and strength profile of outer shaft 404.

FIG. 4C shows an alternative shaft portion 440, which may be used as at least a portion of proximal section 32 of shaft 14. Shaft portion 440 may include an inner shaft 442, which may have any of the properties of inner shaft 402. Inner shaft 442 may define one or more lumens. For example, as shown in FIG. 4C, inner shaft 442 may define first lumen 441 and second lumen 443. First lumen 441 may receive articulation wire 408. Second lumen 443 may receive an element 445. Element 445 may include, for example, an articulation wire, a cable for a sensor, or tubing defining a lumen for transmitting contrast solution. At least first lumen 441 may extend approximately parallel to a central longitudinal axis of shaft portion 440. In other words, at least first lumen 441 may be offset (i.e., oblique) from a center longitudinal axis of shaft portion 440. Second lumen 443 may also be parallel to and offset from the central longitudinal axis of shaft portion 440.

Coil 444 may surround core inner shaft 442, and may include a flat wire coil. For example, coil 444 may include one or more pieces of flattened metal (e.g., wire made from a material such as Nitinol) that are wound to form a coil. A braid 446 may surround coil 444. Braid 446 may include, for example, metal wire braided together to form a sheath. A coating 448 may surround braid 446. Coating 448 may be flexible and may be extruded, molded, or otherwise formed outside of braid 446 (e.g., by reflowing). Coating 448 may contact braid 446 such that a material of coating 448 is fixed to (e.g., fills at least some gaps of) braid 446. Coating 448 may include any suitable materials, including, for example, Pebax 72D. Alternatively, coating 448 may be formed separately and subsequently be positioned around braid 446. Taken together, inner shaft 442, coil 444, braid 446, and coating 448 may give shaft portion 440 a desired combination of flexibility for working through tortuous passages and rigidity for transmitting longitudinal and rotational force.

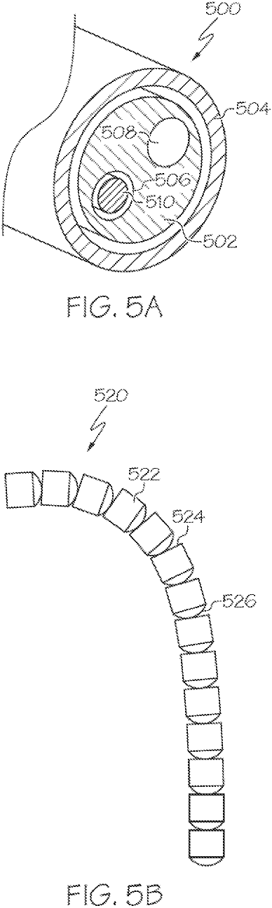

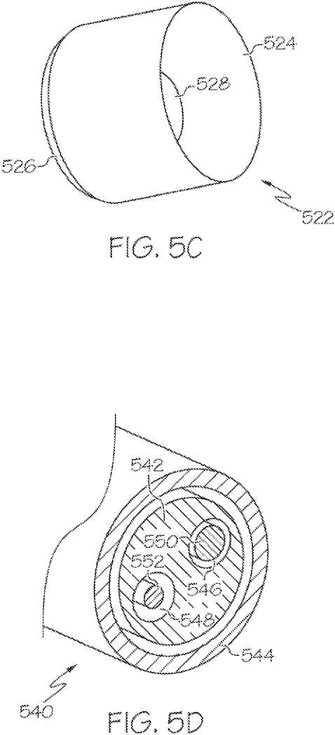

FIGS. 5A-5D show features of exemplary shaft sections that may be used as distal section 34 of shaft 14. Distal section 34 may have features that differ from proximal section 32. For example, distal section 34 may be relatively more flexible than proximal section 32, to allow for articulation of distal section 34 (e.g., via movement of articulation wire 30, as described below). Alternatively, distal section 34 and proximal section 32 may be identical or may have a combination of the features described above and below, with respect to FIGS. 4A-5D. Although two sections (distal and proximal) of shafts are discussed below, it will be appreciated that shaft 14 may have multiple different sections having different properties. For example, there may be a transition on shaft 14 from a more rigid proximal portion 32 to a more flexible distal section 34. Different sections of shaft 14 may be fixed to one another via any suitable method.

A distal end of articulation wire 30 (or any of the articulation wires disclosed herein) may be secured to distal section 34. For example, articulation wire 30 (or another articulation wire) may be welded to distal section 34 or secured by alternative means (e.g., glue or a press-fit). Therefore, the distal end of articulation wire 30 (or another articulation wire) may be fixed relative to distal section 34, but more proximal sections of articulation wire 30 (or another articulation wire) may be movable relative to shaft 14 (including at least portions of proximal section 32 and/or distal section 34).

As shown in FIG. 5A, a shaft portion 500 may include a core inner shaft 502 and an outer shaft 504. Inner shaft 502 may be molded, extruded, or otherwise formed so that it is a single, integral structure that defines one or more lumens. For example, as shown in FIG. 5A, inner shaft 502 may define two lumens. A first lumen 506 may house an articulation wire 510 (which may be used as articulation wire 30 discussed above). As discussed above, a distal end of articulation wire 510 may be secured (e.g., welded) to shaft portion 500 (e.g., a distal portion of shaft portion 500). A second lumen 508 may be used to house a second articulation wire (not shown), or a cable for a sensor (such as an electromagnetic sensor, described below). Additionally or alternatively, second lumen 508 may, directly or via a separate tube within second lumen 508, transmit contrast (e.g., from contrast delivery device 16). At least first lumen 506 may extend approximately parallel to a central longitudinal axis of shaft portion 500. At least first lumen 506 may be offset (i.e., oblique) from a central longitudinal axis of shaft portion 500. Second lumen 508 may also be offset from the central longitudinal axis of shaft portion 500. Because first lumen 506 may be offset from the central longitudinal axis of shaft portion 500, a single articulation wire 510 may serve to articulate shaft portion 500 in two directions (e.g., left and right or up and down). For example, when articulation wire 510 is in a neutral configuration, shaft portion 500 may be approximately straight. When a proximal end of articulation wire 510 is pulled proximally, shaft portion 500 may bend in a first direction. When a proximal end of articulation wire 510 is moved distally, shaft portion 500 may bend in a second direction, opposite to the first direction.

Inner shaft 502 may include any suitable material, such as, for example, Pebax 32D. Portions of inner shaft 502 may also include PTFE, overlapped with Pebax 32D, particularly at a transition point between shaft portion 500 and a more proximal shaft portion (e.g., inner shaft 402 may be adjacent inner shaft 502, and near a boundary therebetween, Pebax 32D and PTFE may be overlaid on one another). Outer shaft 504 may be molded, extruded, or constructed by alternative methods. Outer shaft 504 may be formed atop of inner shaft 502 or separately from inner shaft 502. Outer shaft 504 may include, for example, Pebax 32D or Pebax 35D. Outer shaft 504 may also include varying materials to cause a gradual transition from a stiffer to a more flexible outer shaft 504. For example, a more proximal portion of shaft 504 may include Pebax 55D. Shaft portion 500 may also include a braid, such as braid 446, described above, which may facilitate torsion (rotation) of shaft portion 500 about its longitudinal axis.

FIGS. 5B-5C show aspects of an alternative shaft portion 520. Shaft portion 520 may include a plurality of links 522. Each of links 522 may have a proximal end 524 and a distal end 526. Proximal end 524 may have a concave shape, and distal end 526 may have a convex shape. Alternatively, proximal end 524 and distal end 526 may have alternate (e.g., reversed) shapes. Proximal end 524 of a first link may mate with distal end 526 of a second link (the first link may be distal to the second link). Each of links 522 may define an opening 528. Opening 528 may have a center that is offset from a center of link 522, such that opening 528 is oblique. Together, openings 528 of a plurality of links 522 may define a channel for receiving an articulation wire (such as articulation wire 30 or another articulation wire) and any other elements (e.g., a lumen for transmitting contrast solution, sensor cables, or other structures).

FIG. 5D depicts a shaft portion 540 that may have any of the features of shaft portion 500. Shaft portion 540 may include a core inner shaft 542 (having any of the properties of inner shaft 502) and an outer shaft 544 (having any of the properties of outer shaft 504). Inner shaft 542 may define two lumens. A first lumen 546 may house an articulation wire 550 (which may be used as articulation wire 30 discussed above and may have any of the properties of articulation wire 510, above). A second lumen 548 may house an electromagnetic sensor 552, or wires, cables, or other structures related to an electromagnetic sensor. Electromagnetic sensor 552 may include a coil, which may be attached to a distal end of second lumen 548. Wires or cables may run proximally from electromagnetic sensor 552 to a proximal end of shaft 14. A current may be passed through a portion of electromagnetic sensor 552, such as the coil. A device may be used to generate an external magnetic field that senses a position and/or direction of electromagnetic sensor 552. Thus, electromagnetic sensor 552 may assist an operator with positioning and/or moving medical device 10 to a desired location in a body lumen.

An operation of medical device 10 will now be described. An operator may obtain medical device 10 with handle 12 already attached or may assemble handle 12, as described above. Shaft 14 may be inserted into a body lumen of a subject, either directly or through a channel of another medical device (e.g., an endoscope, duodenoscope, colonoscope, ureteroscope, catheter, or other medical device). As discussed above, an outer surface of shaft 14 may be secured by an element of handle 12 (such as collet 28 and locknut screw 204), such that handle 12 is fixed relative to the proximal end of shaft 14. An operator may grip handle 12 in order to navigate shaft 14. An operator may advance handle 12 in order to advance shaft 14 distally or retract shaft 14 proximally. An operator may also rotate handle 12 about a longitudinal axis of handle 12 in order to rotate shaft 14 about a longitudinal axis of shaft 14. An operator may utilize slider 26 in order to articulate a distal end of shaft 14, using articulation wire 30. As discussed above, a distal end of articulation wire 30 may be fixed to a distal end of shaft 14. As discussed above with respect to FIGS. 2A-3J (and FIG. 3E in particular), a proximal end of articulation wire 30 may be fixed relative to plunger 24. An operator may move slider 26 proximally or distally, relative to plunger 24. As slider 26 moves proximally and distally, shaft 14 may move proximally or distally relative to plunger 24, along with slider 26. As slider 26 moves distally, a tension in articulation wire 30 will increase, as a distal force is exerted on the distal end of articulation wire 30 that is fixed to the distal end of shaft 14. Because, as discussed above, a lumen of shaft 14 holding articulation wire 30 is offset from a center longitudinal axis of shaft 14, this increased tension will cause distal section 34 to articulate (i.e. bend) in a first direction. Moving slider 26 a greater distal amount may correspond to increased articulation of distal section 34. In contrast, when slider 26 is moved proximally, a tension in articulation wire 30 may be decreased. As slider 26 is moved proximally, shaft 14 may move to a relaxed configuration (e.g., a configuration in which distal section 34 is not articulated or bent to one side). In some examples, further proximal movement of slider 26 may cause shaft 14 to articulate in a second direction, opposite to the first direction. The operator may continue to move an entirety of handle 12 proximally or distally and to rotate handle 12 along its central longitudinal axis, to achieve three degrees of freedom of the distal end of shaft 14.

After shaft 14 is positioned in a desired location, handle 12 may be removed by completing steps opposite to those described above for assembling handle 12. An operator may use shaft 14 as is typical for a guidewire. Tools, stents, catheters, or other medical devices may be passed over shaft 14. Shaft 14 may be removed from the body lumen or medical device by simply pulling it proximally. Alternatively, handle 12 may be re-assembled on shaft 14 in order to remove or reposition shaft 14 of medical device 10. Thus, medical device 10 provides for steerable positioning of shaft 14, allowing for precise positioning of shaft 14, as well as medical devices or tools passed over shaft 14.

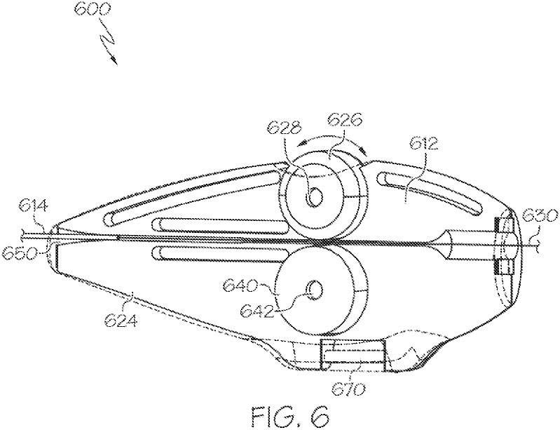

FIG. 6-8B disclose alternative medical devices. Features of the devices shown in FIG. 6-8B may be combined with features of the device(s) described above in any suitable fashion, unless otherwise stated. For example, handle 12, described above, may incorporate any suitable features of FIG. 6-8B.

FIG. 6 shows a medical device 600. FIG. 6 is at least partially transparent to show relevant details internal to medical device 600. Medical device 600 may include a handle 612 and a shaft 614. Shaft 614 may have any of the properties of shaft 14, described above (including the properties described with respect to FIGS. 4A-4C). Handle 612, like handle 12 of medical device 10, may be removable from shaft 614. However, instead of using a slider, an operator may contact a first roller 626, rotatable about an axis 628, in order to articulate shaft 614.

Handle 612 may include a handle housing 624. Handle housing 624 may be made of any suitable material, such as plastic. Handle housing 624 may be molded or otherwise formed so as to have features to receive the structures described herein. A proximal end of shaft 614 may be received within a lumen 650 of handle 612. As shown in FIG. 6, a distal end of lumen 650 may be larger and may taper in a proximal direction. An articulation wire 630 (having any of the properties of articulation wire 30) may extend out of the proximal end of shaft 614, through lumen 650. A proximal end of lumen 650 may also have a wider diameter (e.g., so that contrast or another substance, such as a drug, may be injected through lumen 650), and articulation wire 630 may extend proximally out of a proximal opening of lumen 650. Alternatively, articulation wire 630 may terminate within housing 624 with a length suitable to accommodate proximal and distal movement of articulation wire 630 as first roller 626 moves. Articulation wire 630 may include one or more stops or other features to prevent moving articulation wire proximally or distally past a desired location.

First roller 626 and a second roller 640 (rotatable about a center axis 642) may have outer surfaces that extend into lumen 650. The rollers may contact articulation wire 30 within lumen 650. The outer surface of first roller 626 may also extend outside of handle housing 624. The portion of first roller 626 that extends out of handle housing 624 may be approximately opposite (across a diameter of first roller 626) the portion of first roller 626 that extends into lumen 650. Second roller 640 may be within handle housing 624, such that a surface of second roller 640 does not extend outside of handle housing 624. A user may contact the surface of first roller 626 in order to move first roller 626 in the directions showed by the arrows in FIG. 6. First roller 626 may contact articulation wire 630, and exert a frictional force on articulation wire 630 to move articulation wire 630 proximally or distally. For example, when first roller 626 is moved in the clockwise direction of the vantage point shown in FIG. 6, first roller 626 may exert a distal force on articulation wire 630, moving articulation wire 630 in a distal direction. The distal movement of articulation wire 630 may, in turn, cause second roller 640 to rotate in a counterclockwise direction of the vantage point of FIG. 6. In contrast, when an operator moves first roller 626 in a counterclockwise direction of FIG. 6, first roller 626 may exert a proximal force on articulation wire 630. Articulation wire 630 may, in turn, exert a force on second roller 640 to move second roller 640 in a clockwise direction. While a user may move the distal end of articulation wire 630 using first roller 626, handle housing 624 may retain the proximal end of shaft 614 in a fixed position relative to handle housing 624.

Handle housing 624 may include a hinge 670. 624 may be opened about hinge 670 in order to remove handle housing 624 from shaft 614 (and articulation wire 630). Handle housing 624 may include features (e.g., latches, mating features, or other structures) to maintain handle housing 624 in a closed position when desired by an operator. The operator may be able to unlatch latches or pull apart mating features (or activate other features) in order to open handle housing 624. Handle housing 624 may include distal slots (e.g., semicircular slots) that have a smaller diameter than shaft 614, such that when housing 624 is hinged close, edges of handle housing 624 grip and secure an outside surface of shaft 614 relative to handle housing 624.

An operation of medical device 600 will now be described. An operator may obtain medical device 600 with handle 612 already attached to shaft 614 or may assemble handle 612 and shaft 614. Shaft 614 may be inserted into a body lumen of a subject, either directly or through a channel of another medical device (e.g., an endoscope, duodenoscope, colonoscope, ureteroscope, catheter, or other medical device). An operator may grip handle 612 in order to navigate shaft 614. An operator may advance handle 612 in order to advance shaft 614 distally or retract shaft 614 proximally. An operator may also rotate handle 612 about a longitudinal axis of handle 612 in order to rotate shaft 614 about a longitudinal axis of shaft 614.

An operator may move first roller 626 in order to articulate a distal end of shaft 614, using articulation wire 630. A distal end of articulation wire 630 may be fixed to a distal end of shaft 614. An operator may rotate first roller 626 to move a distal end of articulation wire 630 proximally or distally. As first roller 626 moves counterclockwise in FIG. 6, a tension in articulation wire 630 will increase. Because a lumen of shaft 614 holding articulation wire 630 is offset from a center longitudinal axis of shaft 614, this increased tension will cause a distal section of shaft 614 to articulate (i.e. bend). Greater rotation of first roller 626 may correspond to increased articulation of shaft 614. In contrast, when first roller 626 is rotated clockwise, a tension in articulation wire 630 may be decreased. As first roller 626 is rotated clockwise, shaft 614 may move to a relaxed configuration (e.g., a configuration in which shaft 614 is not articulated or bent to one side). The operator may continue to move an entirety of handle 612 proximally or distally and to rotate handle 612 along its central longitudinal axis, to achieve three degrees of freedom of the distal end of shaft 614.

After shaft 614 is positioned in a desired location, handle 612 may be removed by unhinging handle 612. An operator may use shaft 614 as is typical for a guidewire. Tools, stents, catheters, or other medical devices may be passed over shaft 614. Shaft 614 may be removed from the body lumen or medical device by simply pulling it proximally. Alternatively, handle 612 may be re-assembled on shaft 614 in order to remove or reposition shaft 614 of medical device 600. Thus, medical device 600 provides for steerable positioning of shaft 614, allowing for precise positioning of shaft 614, as well as medical devices or tools passed over shaft 614.

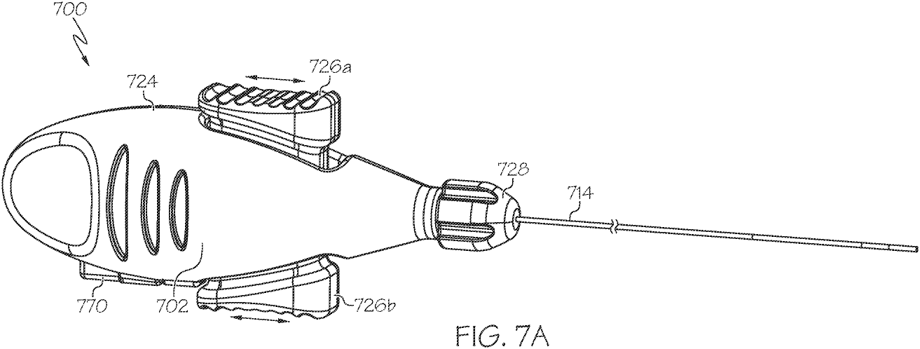

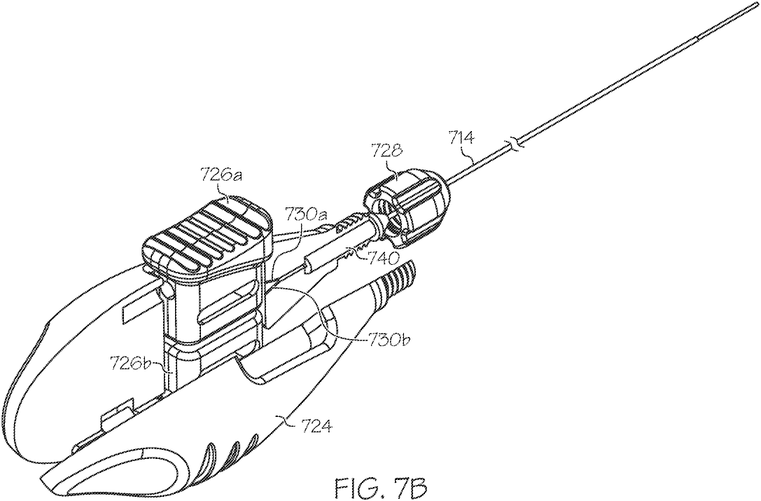

FIGS. 7A and 7B depict a medical device 700. FIG. 7A shows medical device 700 in an assembled state, and FIG. 7B shows medical device 700 in a partially disassembled state. Medical device 700 may have features of one or both of medical device 10 and medical device 600. Medical device 700 may include a handle 712, having a handle body 702, and a shaft 714. Shaft 714 may have any of the properties of shaft 14, described above (including the properties described with respect to FIGS. 4A-4C). Handle 712, like handles 12 and 612, may be removable from shaft 714. Like handle 612, handle 712 may have a housing 724 that includes a hinge 770, which allows housing 724 to be opened and closed. An operator may contact sliders 726a, 726b in order to articulate a distal end of shaft 714.

Two articulation wires 730a, 730b, may extend from a distal end of shaft 714 to, and proximally out of, a proximal end of shaft 714. Articulation wires 730a, 730b may extend through respective lumens offset from a central axis of shaft 714. Proximal ends of articulation wires 730a, 730b may be received and secured within sliders 726a, 726b, respectively. In an example, articulation wires 730a, 730b may have proximal ends having larger diameters (e.g., protrusions), which may be inserted into slots on sliders 726a, 726b, respectively. As shown by the arrows in FIG. 7A, sliders 726a, 726b may be movable in proximal and distal directions to move the proximal ends of articulation wires 730a, 730b proximally and distally, respectively. Sliders 726a, 726b may be independently movable, such that one of articulation wires 730a, 730b may be moved without moving the other of articulation wires 730a, 730b.

A distal end of handle 712 may include a collet 740 and a screw cap 728. Collet 740 may include slits formed therein (e.g., four slits). When screw cap 728 is screwed onto a distal end of housing 724, a tapered profile of screw cap 728 may compress portions of collet 740 between the slits closer to one another, so as to grip onto a proximal end of shaft 714 in order to fix the proximal end of shaft 714 relative to housing 724. Alternatively, a hinged cam-locking hinge or latch (descried above with respect to medical device 10) may be utilized.

To assemble medical device 700 (attach handle 712 to shaft 714), a proximal end of shaft 714 may be inserted into collet 740, and proximal ends of articulation wires 730a, 730b may be inserted into slots in sliders 726a, 726b. Housing 724 may be hinged closed about hinge 770. Screw cap 728 may be threaded over a distal end of shaft 714 and screwed over collet 740. In order to detach handle 712 from shaft 714, the reverse steps may be completed.

An operation of medical device 700 will now be described. An operator may obtain medical device 700 with handle 712 already attached to shaft 714 or may assemble handle 712 and shaft 714. Shaft 714 may be inserted into a body lumen of a subject, either directly or through a channel of another medical device (e.g., an endoscope, duodenoscope, colonoscope, ureteroscope, catheter, or other medical device). An operator may grip handle 712 in order to navigate shaft 714. An operator may advance handle 712 in order to advance shaft 714 distally or retract shaft 714 proximally. An operator may also rotate handle 712 about a longitudinal axis of handle 712 in order to rotate shaft 714 about a longitudinal axis of shaft 714.

An operator may move slider 726a to articulate shaft 714 in a first direction and may move slider 726b to articulate shaft 714 in a second direction, different from the first direction. A distal end of articulation wires 730a, 730b may be fixed to a distal end of shaft 714. As an operator moves slider 726a or 726b proximally, a tension in articulation wire 730a or 730b will increase. Because lumens of shaft 714 holding articulation wires 730a, 730b are offset from a center longitudinal axis of shaft 714, this increased tension will cause a distal section of shaft 714 to articulate (i.e. bend). Greater movement of slider 726a or 726b may correspond to increased articulation of shaft 714. In contrast, when slider 726a or 726b is moved distally, a tension in articulation wire 730a or 730b may be decreased, relaxing shaft 714. The operator may continue to move an entirety of handle 712 proximally or distally and to rotate handle 712 along its central longitudinal axis, to achieve three degrees of freedom of the distal end of shaft 714.

FIGS. 8A and 8B depict a handle 812 of a medical device 800. FIG. 8A shows closed handle 812, and FIG. 8B shows partially transparent handle 812 in order to illustrate internal details of handle 812. Medical device 800 may have features of any of medical devices 10, 600, or 700. Handle 812 may receive a shaft (not shown), which may have any of the properties of shaft 14, described above (including the properties described with respect to FIGS. 4A-4C). Handle 812, like handles 12, 612, and 712 may be removable from a shaft. Like handles 612 and 712, handle 812 may have a housing 824 that includes a hinge 870, which allows housing 824 to be opened and closed. An operator may contact sliders wheels 826a, 826b about axis 827 in order to articulate a distal end of the shaft.

Handle 812, like handle 712, may be configured to receive two articulation wires extending extend from a proximal end of a shaft (not shown). Handle 812 may include lumens 832a, 832b for receiving the respective articulation wires. Terminal ends 834a, 834b of lumens 832a, 832b may be disposed within or otherwise fixed to wheels 826a, and 826b, respectively. Proximal ends of the two articulation wires may be fixed to wheels 826a, 826b, respectively (one wire per wheel). The proximal ends of the articulation wires may have an increased diameter (e.g., a protruding portion) that mates with lumens 832a, 832b to frictionally fix the proximal ends of the articulation wires relative to lumens 832a, 832b. As shown by the arrows in FIG. 8A, wheels 826a, 826b may be rotatable to move the proximal ends of the articulation wires. When wheels 826a, 826b are moved in a clockwise direction of FIG. 8A, the proximal ends of the articulation wires may be moved distally. In contrast, when wheels 826a, 826b are moved in a counterclockwise direction of FIG. 8B, the proximal ends of the articulation wires may be moved proximally. Wheels 826a, 826b may be independently movable, such that one of the articulation wires may be moved without moving the other of the articulation wires.

Similarly to handle 712, a distal end of handle 812 may include a collet 840 and a screw cap 828, which may have any of the properties of collet 740 and screw cap 728, respectively. As described above for medical device 700, collet 840 and screw cap 828 may cooperate to grip and fix a proximal end of the shaft. As described above with respect to medical device 10, a hinged cam-locking hinge or latch may be used as an alternative to screw cap 828.

An operation of medical device 800 will now be described. An operator may obtain medical device 800 with handle 812 already attached to the shaft (not shown but having any properties of the other shafts described herein) or may assemble handle 812 and the shaft. The shaft may be inserted into a body lumen of a subject, either directly or through a channel of another medical device (e.g., an endoscope, duodenoscope, colonoscope, ureteroscope, catheter, or other medical device). An operator may grip handle 812 in order to navigate the shaft. An operator may advance handle 812 in order to advance the shaft distally or retract the shaft proximally. An operator may also rotate handle 812 about a longitudinal axis of handle 812 in order to rotate the shaft about a longitudinal axis of the shaft.

An operator may rotate wheel 826a to articulate the shaft in a first direction and may rotate wheel 826b to articulate the shaft in a second direction, different from the first direction. A distal end of the articulation wires may be fixed to a distal end of the shaft. As an operator rotates wheels 826a or 826b in a counterclockwise direction of FIG. 8A, a tension in the articulation wire will increase, thereby articulating the shaft. Greater movement of wheel 826a or 826b may correspond to increased articulation the shaft. In contrast, as wheel 826a or 826b is rotated in a clockwise direction of FIG. 8B, a tension in the articulation wire may be decreased, relaxing the shaft. The operator may continue to move an entirety of handle 812 proximally or distally and to rotate handle 812 along its central longitudinal axis, to achieve three degrees of freedom of the distal end of the shaft.

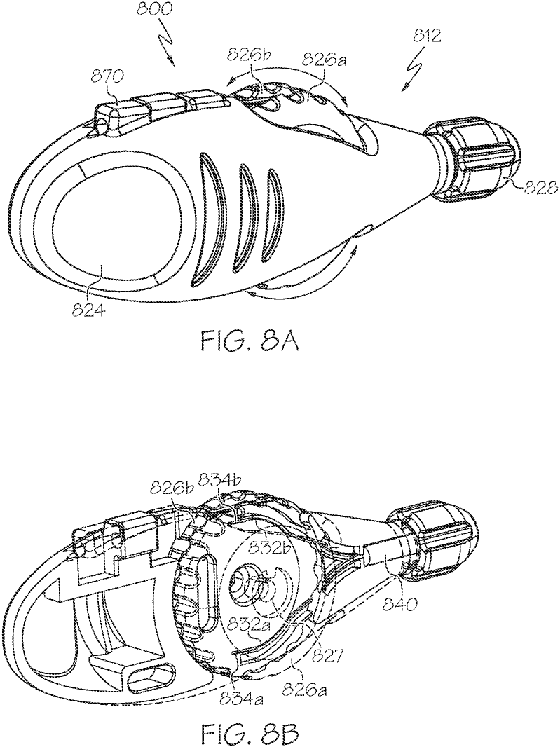

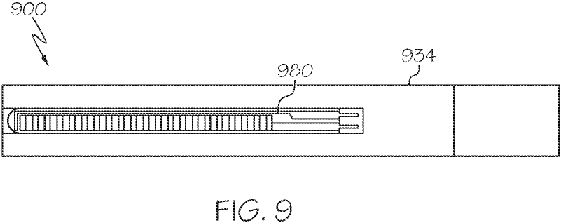

FIG. 9 shows an example portion 934 of a shaft 900, features of which may be used in conjunction with any of the devices described herein. Portion 934 may be a distal portion of shaft 900. Portion 934 may include a flex sensor 980. Flex sensor 980 may measure an amount of deflection or bending of portion 934. Flex sensor 980 may be adhered to a surface of portion 934. Resistance of flex sensor 980 may vary as portion 934 bends/is articulated. A change in resistance of flex sensor 980 may be measured by a controller in terms of a voltage, which may correlate to an amount (e.g., in degrees) portion 934 is bent. Software of the controller may measure the voltage and provide details via a user interface as to the shape of portion 934. Thus, flex sensor 980 may facilitate positioning of shaft 900 in a desired location.

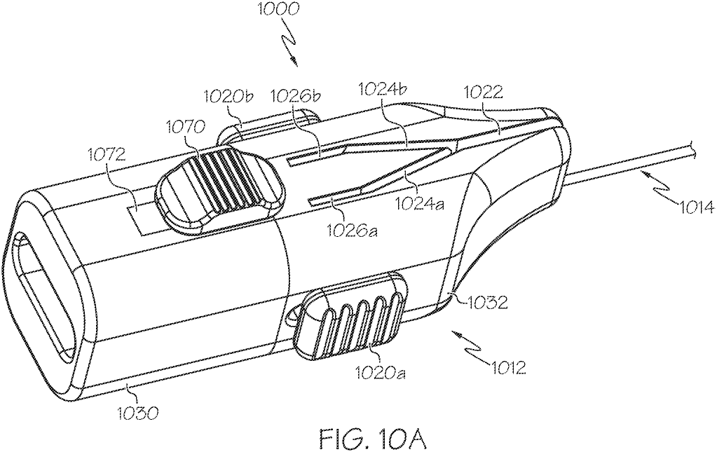

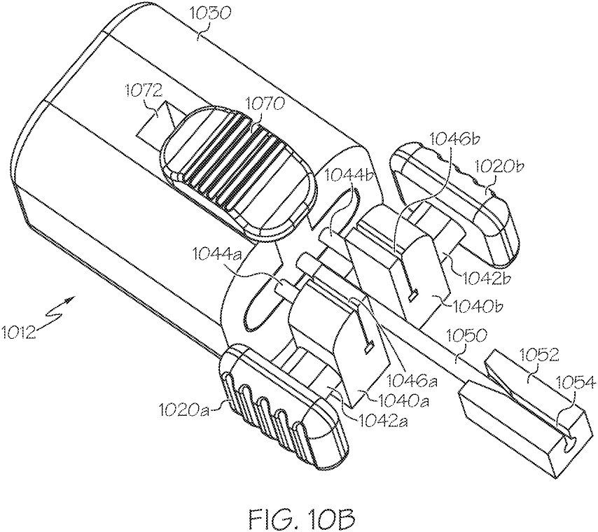

FIGS. 10A-10C depict another example device 1000. FIGS. 10B and 10C depict device 1000 with certain components removed in order to show internal features of device 1000. Features of device 1000 may be combined in any suitable manner with the other devices described herein. With reference to FIG. 10A, device 1000 may include a handle 1012 and a shaft 1014. Handle 1012 may include two steering sliders 1020a, 1020b. Although two steering sliders 1020a, 1020b are depicted, it will be appreciated that alternative numbers of sliders (e.g., one slider, or more than two sliders) may be utilized. Other control members (e.g., knobs, levers, joysticks, or buttons) may also be used. Handle 1012 includes features that facilitate attaching and detaching shaft 1014 from handle 1012. For example, a first body portion 1032 of handle 1012 may define a forked channel (or slot) 1022, which may be configured to receive and release steering components of shaft 1014, described below. Forked channel 1022 may have an approximately Y-shape. The Y-shape may be suited accommodate shaft 1014 in the stem of the “Y” shape (the single channel) and articulation wires 1060a, 1060b, as discussed further below. A distal portion of channel 1022 may include only one channel. More proximally, channel 1022 may include two forked portions 1024a, 1024b, which may extend transversely to a longitudinal axis of handle 1012. Proximalmost sections 1026a, 1026b of channel 1022 may extend proximally from forked portions 1024a, 1024b, respectively. Proximalmost sections 1026a, 1026b may extend approximately parallel to the longitudinal axis of handle 1012. A second body portion 1030 of handle 1012 (proximal of first body portion 1032) may include a control member such as a release slider 1070, slidably received within a slot 1072 defined by second body portion 1030.

FIGS. 10B and 10C depict handle 1012 with first body portion 1032 removed, in order to depict internal mechanisms of handle 1012. FIG. 10B shows handle 1012 without shaft 1014 removably attached, and FIG. 10C shows shaft 1014 removably attached to handle 1012. As shown in FIGS. 10B and 10C, sliders 1020a and 1020b may extend laterally from wire fixation bodies 1040a, 1040b, respectively, housed within first body portion 1032 in FIG. 10A. Neck portions 1042a, 1042b (having narrower dimensions than sliders 1020a and 1020b and wire fixation bodies 1040a, 1040b) may extend between sliders 1020a and 1020b and wire fixation bodies 1040a, 1040b, respectively.

Wire fixation bodies 1040a, 1040b may define adjustable wire slots 1046a, 1046b, respectively. As discussed in further detail below, wire slots 1046a, 1046b may be configured to removably receive articulation wires 1060a, 1060b, respectively of shaft 1014 (see FIG. 10C). Wire slots 1046a, 1046b may extend through a length of wire fixation bodies 1040a, 1040b, from proximal ends of wire fixation bodies 1040a, 1040b to distal ends of wire fixation bodies 1040a, 1040b, respectively. Wire slots 1046a, 1046b may extend approximately parallel to the longitudinal axis of handle 1012. Wire slot 1046a may be aligned with a portion of channel 1022, such as proximalmost section 1026a of channel 1022. Wire slot 1046b may be aligned with a portion of channel 1022, such as proximalmost section 1026b of channel 1022.

Lumens 1044a, 1044b may extend proximally from wire fixation bodies 1040a, 1040b, respectively, though a proximal wall of second body portion 1030. Lumens 1044a, 1044b may carry control mechanisms (e.g., cables or shafts) between release slider 1070 and wire fixation bodies 1040a, 1040b, respectively. As discussed in further detail below, movement of release slider 1070 proximally or distally may cause wire slots 1046a, 1046b to widen or narrow. The control mechanism(s) may transfer the forces necessary to cause such widening or narrowing of wire slots 1046a, 1046b (in a direction perpendicular to an axis of wire slots 1046a, 1046b), upon proximal or distal movement of slider 1070 within slot 1072. For example, the control mechanisms may cause portions of walls defining wire slots 1046a, 1046b to extend inward toward a central longitudinal axis of wire slots 1046a, 1046b.

Internally of first body portion 1032, handle 1012 may also include a shaft fixation body 1052, which may define a shaft slot 1054. Shaft slot 1054 may be configured to removably receive a proximal portion of shaft 1014 (see FIG. 10C). Shaft slot 1054 may open wider than wire slots 1046a, 1046b, to accommodate a greater width of shaft 1014 as compared to articulation wires 1060a, 1060b. Shaft slot 1054 may extend approximately parallel to the longitudinal axis of handle 1012. Shaft slot 1054 may be aligned with a portion of channel 1022, such as a distal section of channel 1022 (a portion of channel 1022 that is not forked).

A lumen 1050 may extend proximally from shaft fixation body 1052, through a proximal wall of second body portion 1030. Lumen 1050 may carry control mechanisms (e.g., cables or shafts) between release slider 1070 and shaft fixation body 1052. As discussed in further detail below, movement of release slider 1070 proximally or distally may cause shaft slot 1054 to widen or narrow, in a direction perpendicular to a longitudinal axis of shaft slot 1054. The control mechanism(s) may transfer the forces necessary to cause such widening or narrowing of shaft slot 1054, upon proximal or distal movement of slider 1070 within slot 1072. For example, the control mechanisms may cause portions of walls shaft slot 1054 to extend inward toward a central longitudinal axis of shaft slot 1054.

Shaft slot 1054 may narrow a relatively smaller amount as compared to wire slots 1046a, 1046b to accommodate a larger width of shaft 14 as compared to articulation wires 1060a, 1060b. When shaft slot 1054 is in a narrow (or, in other words, closed) configuration, it may have a same or smaller width than the width of a proximal portion of shaft 14, so that shaft slot 1054 frictionally retains shaft 14 therein. When wire slots 1046a, 1046b are in a narrow (or, in other words, closed) configuration, they may have a same or smaller width than the width of proximal portions of articulation wires 1060a, 1060b, so that wire slots 1046a, 1046b frictionally retain articulation wires 1060a, 1060b, respectively, therein. In contrast, when shaft slot 1054 is in a wider (or, in other words, open) configuration, it may have a larger width than the width of a proximal portion of shaft 14, so that shaft 14 may be removed from shaft slot 1054. When wire slots 1046a, 1046b are in a wider (or, in other words, open) configuration, they may have a larger width than the width of proximal portions of articulation wires 1060a, 1060b, so that articulation wires 1060a, 1060b may be removed from wire slots 1046a, 1046b, respectively.

Each of FIGS. 10A-10C depicts slider 1070 in a distal position (in a first configuration), which may correspond to a position in which wire slots 1046a, 1046b and shaft slot 1054 are in an open position. In the depicted configuration, a proximal portion of shaft 1014 may be inserted through channel 1022, into shaft slot 1054. Proximal portions of articulation wires 1060a, 1060b (which may have any of the properties of the articulation wires described above) may extend proximally past a proximal end of shaft 1014. The proximal portions of articulation wires 1060a, 1060b may be inserted through proximalmost sections 1026a, 1026b, into wire slots 1046a, 1046b, respectively, seating them therein. Shaft 1014, articulation wires 1060a, 1060b, shaft slot 1054, and wire slots 1046a, 1046b may include features that facilitate seating shaft 1014 within shaft slot 1054 and articulation wires 1060a, 1060b within wire slots 1046a, 1046b, respectively. Such features may include, for example, mating features, grooves, and/or protruding portions.

Thereafter, slider 1070 may be moved proximally to a second configuration, narrowing wire slots 1046a, 1046b and shaft slot 1054. As wire slots 1046a, 1046b and shaft slot 1054 narrow, they may tighten their grip (exert a radially inward force) on shaft 1014 and articulation wires 1060a, 1060b, respectively. Shaft 1014 may thus be fixedly secured relative to shaft slot 1054 and shaft fixation body 1052. Articulation wires 1060a, 1060b may thus be fixedly secured relative to wire slots 1046a, 1046b and wire fixation bodies 1040a, 1040b, respectively. Moving slider 1070 proximally may thereby serve to operatively secure shaft 1014 and articulation wires 1060a, 1060b to handle.

The relative motion of slider 1070 (proximal or distal) is not limited by the disclosure. For example, a proximal movement of slider 1070 may serve to open wire slots 1046a, 1046b and shaft slot 1054, and distal movement of slider 1070 may serve to narrow wire slots 1046a, 1046b and shaft slot 1054. Slider 1070 is merely exemplary, and any suitable control may be used (e.g., one or more knob, lever, joystick, or button).

When shaft 1014 (and articulation wires 1060a, 1060b) are secured to handle 1012, device 1000 may be operated to perform a medical procedure. An operator may obtain medical device 1000 with handle 1012 already attached to shaft 1014 or may assemble handle 1012 and shaft 1014, as described above. Shaft 1014 may be inserted into a body lumen of a subject, either directly or through a channel of another medical device (e.g., an endoscope, duodenoscope, colonoscope, ureteroscope, catheter, or other medical device). An operator may grip handle 1012 in order to navigate shaft 1014. An operator may advance handle 1012 in order to advance shaft 1014 distally or retract shaft 1014 proximally. An operator may also rotate handle 1012 about a longitudinal axis of handle 1012 in order to rotate shaft 1014 about a longitudinal axis of the shaft.

An operator move slider 1020a proximally or distally to move articulation wire 1060a (fixed to slider 1020a via wire fixation body 1040a) proximally or distally, respectively. An operator move slider 1020b proximally or distally to move articulation wire 1060b (fixed to slider 1020b via wire fixation body 1040b) proximally or distally, respectively. Distal ends of articulation wires 1060a, 1060b may be fixed to a distal end of shaft 1014. Thus, proximal and distal movement of sliders 1020a, 1020b may cause articulation of a distal portion of shaft 1012.The operator may continue to move an entirety of handle 1012 proximally or distally and to rotate handle 1012 along its central longitudinal axis, to achieve three degrees of freedom of the distal end of shaft 1014.

Any of the handles disclosed herein may be removed and/or replaced with the same handle or with an alternate handle. A removable and/or replaceable handle may be particularly advantageous because, in complex ERCP procedures in the intrahepatic ducts, for example, an operator may need to access multiple sites in the hylar tree during the procedure. The disclosed removable and/or replaceable handles may facilitate such repositioning without retracting an entire guidewire (i.e., shaft) and re-introducing a new guidewire (i.e., shaft). Alternatively, an entirety of the devices may be disposable (or reusable). With a replaceable handle, an operator may steer the guidewire to a new location during the same procedure where difficult access has previously been established. Furthermore, removable and/or replaceable handles may support a device that is partially disposable and partially reusable. For example, a shaft portion of the device may be disposable, while the handle portion of the device may be reusable. Additionally or alternatively, multiple different handles may be utilized during one procedure. The devices of this disclosure may have an advantage of providing multiple degrees of freedom of movement-including articulation of the shaft in one or more directions, rotating the shaft, and moving the shaft axially (proximally and/or distally). The disclosed devices may support cost-effective manufacture, due to a low number of components and a low number of elements to assemble within the handle to achieve the degrees of freedom.

Similarly, the shafts disclosed herein may be utilized with any of the disclosed handles. Thus, during a procedure, an operator may use different handles at different portions of the procedure or may use a shaft without a handle. The handles and shafts disclosed herein may also include any suitable combination of features (e.g., one or two articulation wires, location of articulation wires within the shaft, shaft materials, securing mechanisms, etc.).

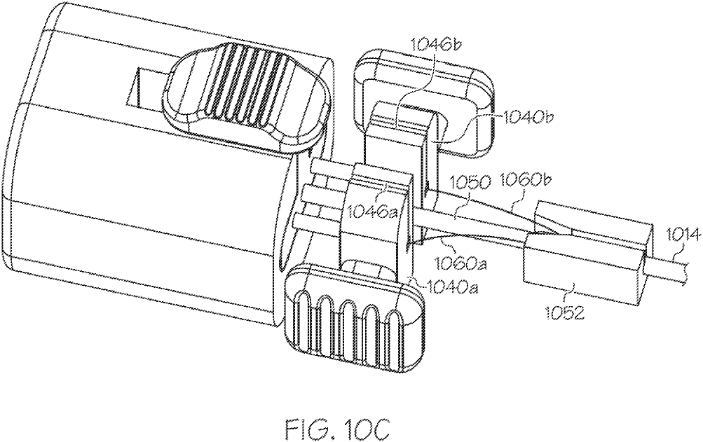

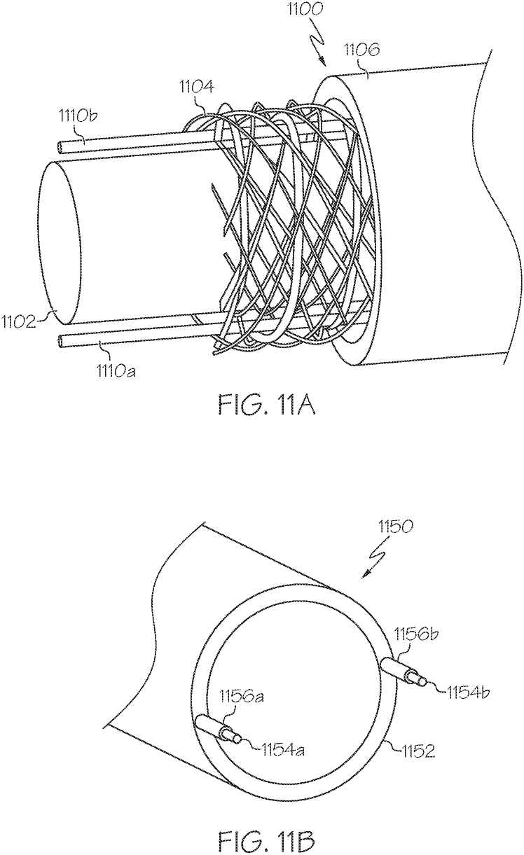

FIGS. 11A-11B depict alternative shafts 1100 and 1150 that may be used with any of the handles disclosed herein. Shaft 1100 (shown with layers thereof progressively removed to show inner layers) may include an inner liner 1102 (e.g., a PTFE liner), which may define a central lumen. Devices or substances (e.g., contrast) may be passed through the lumen defined by inner liner 1102. A braid 1104 (having any of the properties of braid 446, described above) may surround inner liner 1102. Articulation wires 1110a, 1110b may pass between braid 1104 and inner liner 1102. Articulation wires 1110a, 1110b may have any of the properties of the other articulation wires disclosed herein and may be fixedly connected to a distal portion of shaft 1100. An outer shaft (e.g., including resin) 1106 may surround braid 1104. The materials and configurations of shaft 1100 are merely exemplary. Features of shaft 1100 may apply to other sheaths disclosed herein. For example, the shafts of FIGS. 4A-5D may include articulation wires extending between layers of the shafts.

FIG. 11B depicts another example shaft 1150 (shown with layers thereof progressively removed to show inner layers), which may have any of the properties of the other shafts disclosed herein. Shaft 1150 may include a sleeve 1152 including any suitable material (such as the materials discussed above for the shafts of FIGS. 4A-5D and 11A). Articulation wires 1154a, 1154b may pass through lumens defined in sleeve 1152. Articulation wires 1154a, 1154b may be surrounded by sleeves 1156a, 1156b, respectively, to facilitate articulation of articulation wires 1154a, 1154b. Features of shafts 1150 may be used in any of the shafts of FIGS. 4A-5D and 11A. For example, outer shaft 404 (FIG. 4A), coating 448 (FIG. 4C), outer shaft 504 (FIG. 5A), outer shaft 544 (FIG. 5D), or outer shaft 1106 (FIG. 11A) may define lumens through which articulation wires (and, optionally, sleeves) may pass.

While principles of this disclosure are described herein with the reference to illustrative examples for particular applications, it should be understood that the disclosure is not limited thereto. Those having ordinary skill in the art and access to the teachings provided herein will recognize additional modifications, applications, and substitution of equivalents all fall within the scope of the examples described herein. Accordingly, the invention is not to be considered as limited by the foregoing description.

We claim:

1. A medical device comprising:
a shaft;
an articulation wire extending through at least a portion of the shaft; and
a handle, including:
a handle body having an outer surface that defines a channel, wherein a distal portion of the shaft is received within a first portion of the channel, and wherein a distal portion of the articulation wire is received within a second portion of the channel; and
a control member that is movable relative to the handle body to move the distal portion of the articulation wire relative to the distal portion of the shaft.

2. The medical device of claim 1, wherein the articulation wire is a first articulation wire, and the control member is a first control member, and further comprising:
a second articulation wire extending through the at least the portion of the shaft, and wherein a proximal portion of the second articulation wire is removably received within a third portion of the channel; and
a second control member that is movable relative to the handle body to move the distal portion of the second articulation wire relative to the distal portion of the shaft.

3. The medical device of claim 2, wherein each of the second portion of the channel and the third portion of the channel is proximal to the first portion of the channel.

4. The medical device of claim 3, wherein the channel has an approximately Y-shape.

5. The medical device of claim 1, further comprising a wire fixation body connected to the control member, wherein the wire fixation body defines a first slot in communication with the second portion of the channel, and wherein the articulation wire is removably received within the first slot.

6. The medical device of claim 5, wherein the control member is a first control member, and further comprising a second control member, wherein movement of the second control member relative to the handle body changes a width of the first slot.

7. The medical device of claim 6, further comprising a shaft fixation body defining a second slot in communication with the first portion of the channel, wherein movement of the second control member relative to the handle body changes a width of the second slot.

8. The medical device of claim 7, wherein movement of the second control member in a first direction relative to the handle body widens the first slot and the second slot so that the shaft is removable from the second slot and the articulation wire is removable from the first slot.

9. The medical device of claim 8, wherein movement of the second control member in a second direction relative to the handle body narrows the first slot and the second slot so that the articulation wire is fixedly secured relative to the first slot and the shaft is fixedly secured relative to the second slot.

10. The medical device of claim 7, wherein a first lumen extends proximally from the wire fixation body, and wherein a second lumen extends proximally from the shaft fixation body.

11. The medical device of claim 7, wherein the shaft fixation body is distal to the wire fixation body.

12. The medical device of claim 6, wherein the second control member includes a slider.

13. The medical device of claim 6, wherein the first control member is disposed on a first side of the handle, and wherein the second control member is disposed on a second side of the handle, wherein the second side is different from the first side.

14. The medical device of claim 13, wherein the channel is on the first side of the handle.

15. The medical device of claim 1, wherein the articulation wire extends proximally of a proximal end of the shaft.

16. A medical device comprising:
a shaft;
a first articulation wire extending through at least a portion of the shaft;
a second articulation wire extending through at least the portion of the shaft; and
a handle, including:
a first wire fixation body defining a first slot, wherein a proximal end of the first articulation wire is received within the first slot;
a second wire fixation body defining a second slot, wherein a proximal end of the second articulation wire is received within the second slot; and
a control member, wherein movement of the control member changes a first width of the first slot and a second width of the second slot.

17. The medical device of claim 16, further comprising a shaft fixation body defining a third slot, wherein the shaft is received within the third slot, and wherein the movement of the control member changes a third width of the third slot.

18. The medical device of claim 16, wherein the control member is a first control member, wherein the medical device further comprises:
a second control member configured to move the first wire fixation body, thereby moving the proximal end of first articulation wire relative to the shaft; and
a third control member configured to move the second wire fixation body, thereby moving the proximal end of the second articulation wire relative to the shaft.

19. A method of using a medical device, the method comprising:
inserting a proximal end of a shaft into a first portion of a channel defined by a body of a handle of the medical device;
inserting a proximal end of a first articulation wire into a second portion of the channel;
inserting a proximal end of a second articulation wire into a third portion of the channel; and
moving a control member in a first direction relative to the body, thereby securing the shaft, the first articulation wire, and the second articulation wire to the handle.

20. The method of claim 19, the method further comprising:
moving a control wire in a second direction relative to the body, thereby releasing the shaft, the first articulation wire, and the second articulation wire from the handle;
removing the shaft, the first articulation wire, and the second articulation wire from the handle.
